(19) United States
(12) Patent Application Publication
(10) Pub. No.: US 2003/0026827 A1
(43) Pub. Date: Feb. 6, 2003

(54) Additives for making novelty edible comestibles and scented products that are designed for sexual arousal

(76) Inventor: Mark Moore, Tallahassee, FL (US)

Correspondence Address: Carnes, Cona & Dixon, Innovation Park, 1673 West Paul Dirac Drive, Tallahassee, FL, 32310-3763, US

(21) Appl. No.: 10/153,573

(22) Filed: May 22, 2002

Related U.S. Application Data
(60) Provisional application No. 60/293,484, filed on May 25, 2001.

(51) Int. Cl. A61K 9/68

(52) U.S. Cl. 424/440

(57) ABSTRACT

The present invention includes additives that are used for making novelty edible comestibles and/or scented products. The additives are specifically geared towards men and women, so as to provide for a final product that is exciting to utilize and one that will produce favorable results. The additives added have medically been proven to significantly increase penile blood flow in men and increase vaginal blood flow. This increase in blood flow in both men and women is an indication of sexual arousal.

In adding the particular additive to a novelty edible and/or consumable comestibles provides for a product that is fun and easy to use. The edible and/or consumable comestibles provide a means of excitement and will leave an aroma that has medically been proven to increase sexual desired.

In addition, these additives can be added to other items, such as candles, air fresheners, potpourri, or the like. Thus, giving an entire room/area a continuous flow of a particular scent that can stimulate sexual desire.

Accordingly, it is an object of the present invention to provide additives to conventional items that can increase vaginal blood flow in women and/or increase penile blood flow in men to inherently provide for a final product that will increase sexual desires.

It is another object of the present invention to provide for novelty items that are fun and titillating to use and/or consume and one that may increase sexual desire in men, women, or both men and women simultaneously.

Yet a further object of the present invention, to be specifically enumerated herein, is to provide a additives which address the power of olfaction for increase sexual desire

### BACKGROUND OF THE INVENTION

1. Field of the Invention

The present invention relates generally to additives for making novelty edible comestibles and/or scented products and more particularly to certain additives that have been medically proven to increase sexual desires and arousal.

2. Description of the Prior Art

Sexuality is a passion that is extremely desirable by both the male and female population. Many will utilize products, such as oysters, that have a reputation for being aphrodisiacs in order to enhance as well as provide the environment for sexual enhancement. Though many of these products may be harmless, these items do not have the medical research, which supports their aphrodisiac characteristics.

Knowing the popularity in these items that will enhance and stimulate sexual desires, products have been developed that are specifically designed for aphrodisiacs enhancement. For example in U.S. Pat. No. 5,281,423 there is disclosed an aqueous solution containing hydriodic acid. Though this solution may increase sexual desires, it is limited to the female population. In addition, this product fails to address the olfaction, which is a sense that can aid and entice sexual arousal.

What is needed is an additive that can be incorporated into conventional items, so as to provide for a final product that not only acts as an aphrodisiac, but one that will inexpensive to fabricated and be titillating to the consumer. In addition, this product can also be designed to address olfaction, and thus provide a device that is also enhancing to one's sense of smell.

As will be seen, the present invention achieves its intended purposes, objectives and advantages by accomplishing the needs as identified above, through a new, useful and unobvious combination of component elements, which is exciting to use. In addition, the present invention will offer a means of increase sexual desire with additives that have medically been proven to increase sexual desires in both males and females. and which will conform to conventional forms of manufacture, be of simple construction and easy to use so as to provide a final product that is economically feasible, fun for experimentation and relatively successful during utilization.

Although there have been many inventions related to aphrodisiacs, none of the inventions provide additives that can be added to edible or consumable comestibles so as to provide for the consumer to exert a particular aroma or sense that has sexual stimulation properties. The invention further provides for particular additives to be included in various conventional air freshening elements so as to dispense a particular sexual stimulating sense. The present invention meets the requirements of the simplified design, compact size, low initial cost, low operating cost, ease of installation and maintainability.

The foregoing has outlined some of the more pertinent objects of the invention. These objects should be construed to be merely illustrative of some of the more prominent features and application of the intended invention. Many other beneficial results can be obtained by applying the disclosed invention in a different manner or modifying the invention within the scope of the disclosure. Accordingly, a fuller understanding of the invention may be had by referring to the detailed description of the preferred embodiments.

### DETAILED DESCRIPTION OF THE PREFERRED EMBODIMENT

The present invention includes sexually stimulating additives that are added to conventional products so as to provide for a final novelty item that will increase sexual desires in both men and women. The conventional products, in one embodiment, are preferably in the form of edible or consumable comestibles. This will provide for the additives to be included in any edible food, such as, but not limited to, candy, chips, ice cream, popsicles, bread products, rice products, crackers, or the like. Further that consumable comestibles provides for the additives to be included in conventional drinkable items, such as, but not limited to water, alcohol, juice, sports drinks, soda, breath freshener products (including spray dispensers), gum, or the like.

In essence, the additives provide for the sense of taste as well as the sense of smell to be enticing and thus provide for penile blood flow in men and vaginal blood flow in women to be increased, so as to inherently increase sexual desire. The additives that are utilized are additives that have medically been researched and have proven to be successful in increasing sexual desire in men and women. These additives include for men: lavender, pumpkin pie, doughnut, licorice or a combination thereof These additives include for women: cucumber, Good and Plenty™, baby powder, lavender, pumpkin pie, or a combination thereof Thus, the oil essences for these scents, which are conventional, are added to the conventional edible or consumable comestible. During and after consuming the particular comestible containing the particular additive, the user will be exerting the aroma of the particular additive. This in turn will cause for the individual and/or person(s) surrounding the particular individual to be aroused.

In an alternative embodiment, the additives as defined above can be added to other odor exerting items, such as, but not limited to potpourri, room fresheners, candles, novelty undergarments, contraceptives, lip balm, lotions, cosmetics, or the like. Thus providing for items, such as lipstick, to be scented so as to be sexually arousing. The use of these additives to the products will provide for a playful and fun environment. For example, adding a particular scent to a condom will provide for a moment of pleasure that is heightens by the particular scent. These additives include for men: lavender, pumpkin pie, doughnut, licorice or a combination thereof These additives include for women: cucumber, Good and Plenty™, baby powder, lavender, pumpkin pie, or a combination thereof Thus, the oil essences for these scents, which are conventional, are added to the conventional useable, such as the ones identified above.

In summary, the present invention will provide for numerous products including but not limited to liquid concentrates which may be used as a breath freshener, or the tick-tack type breath freshener, or as nail polish, or providing for the ability to be dispensed in a toy type syringe as a liquid drink.

While the present invention has been particularly shown and described with reference to an embodiment thereof, it will be understood by those skilled in the art that various changes in form and detail may be made without departing from the spirit and scope of the present invention.

I claim:

1. A method of providing for sexually stimulating additives in a male individual comprising:
administering to the male additives that are added to conventional products in an amount effective to increase sexual desires in men;
the additive selected from the group consisting of lavender, or pumpkin pie, or donut, or licorice, or vanilla, or cola, or a combination thereof

2. A method of providing for sexually stimulating additives in a male individual that are added to candy products comprising:
administering to the male by consumption of candy in an amount effective to increase sexual desires in men;
the additives selected from the group consisting of a mixture of lavender, or pumpkin pie or donut, or licorice, or vanilla, or cola, or a combination thereof.

3. A method of providing for sexually stimulating additives in a male individual that are added to candy products comprising:
administering to the male by consumption of candy in an amount effective to increase sexual desires in men;
wherein the candy is selected from the group consisting of a cola flavor, a pumpkin pie flavor, a lavender flavor, a donut flavor, a licorice flavor, a vanilla flavor, and a candy mixture comprising one or more of those flavors.

4. An article of manufacture comprising:
(a) conventional products as recited in claim 1 packaged wherein the conventional product when consumed by a male individual is effective to increase sexual desires; and
(b) instructions for use of the conventional products according to the method of claim 1.

5. An article of manufacture comprising:
(a) a candy as recited in claim 2 packaged, wherein the candy when consumed by a male individual is effective to increase sexual desires; and
(b) instructions for use of the candy according to the method of claim 2.

6. An article of manufacture comprising:
(a) a candy as recited in claim 3 packaged, wherein the candy when consumed by a male individual is effective to increase sexual desires; and
(b) instructions for use of the candy according to the method of claim 3

7. The method of claim 1, wherein the additive is administered in a form selected from the group consisting of an icecream bit, minibottle, liquid concentrate, lipbalm, tic-tac type hardpack, peanut type mini packet, suckers/lollypops, large multiflavored/flavored sucker, gum, nail polish, candy bars, flavored drinks or the like, and flavored scented condoms.

8. The method of claim 1 wherein the additive is administered in a form selected from the group consisting of any edible food such as candy, gum, chips, ice cream, popsicles, bread products, rice products, crackers or the like.

9. The method of claim I wherein the additive is administered in drinkable items such as alcohol, juice, sport drinks, soda, and breath freshener products or the like.

10. The method of claim 1, wherein the additive administered in conventional product forms selected from scents of the group which are added to the conventional products.

11. The method of claim 2, wherein the additive administered in a candy form selected from scents from the group which are added to the candy.

12. A method of providing for sexually stimulating additives in a female individual comprising;
administering to the female additives that are added to conventional products in an amount effective to increase sexual desires in women;
the additive selected from the group consisting of cucumber, Good and Plenty™, baby powder, lavender, pumpkin pie, or a combination thereof.

13. A method of providing for sexually stimulating additives in a female individual that are added to candy products comprising:
administering to the female by consumption of candy in an amount effective to increase sexual desires;
the candy selected from the group consisting of a mixture of cucumber, Good and Plenty ™, baby powder, lavender, pumpkin pie, or a combination thereof.

14. A method of providing for sexually stimulating additives in a female individual that are added to candy products comprising:
administering to the female by consumption of candy in an amount effective to increase sexual desires;
wherein the candy is selected from the group consisting of a cucumber flavor, Good and Plenty™ flavor, a baby powder flavor, a lavender flavor, a pumpkin pie flavor, and a candy mixture comprising one or more of those Flavors.

15. An article of manufacture comprising:
(a) a candy recited in claim 1 packaged, wherein the conventional product when consumed by a female individual is effective to increase sexual desires; and
(b) instructions for use of the conventional products according to the method of claim 1.

16. An article of manufacture comprising:
(a) a candy as recited in claim 2 packaged, wherein the candy when consumed by a female individual is effective to increase sexual desires; and
(b) instructions for use of the candy according to the method of claim 2.

17. An article of manufacture comprising:
(a) a candy as recited in claim 3 packaged, wherein the candy when consumed by a male individual is effective to increase sexual desires; and
(b) instructions for use of the candy according to the method of claim 3.

19. The method of claim 13, wherein the additive is administered in a form selected from the group consisting of cucumber, Good and Plenty ™, baby powder, lavender, pumpkin pie, or a combination thereof.

20. The method of claim 13, wherein the additive is administered in a form selected the group consisting of an icecream bit, minibottle, liquid concentrate, lipbalm, tic-tac type hardpack, peanut type mini packet, suckers/lollypops, large multiflavored/flavored sucker, gum, nail polish, candy bars, flavored drinks or the like, and flavored scented condoms.

21. The method of claim 13, wherein the additive is administered in a form selected from the group consisting of any edible food such as candy, gum, chips, ice cream, popsicles, bread products, rice products, crackers or the like.

22. The method of claim 13, wherein the additive administered in conventional product forms selected from the scents of the group which are added to the conventional products.

23. The method of claim 14, wherein the additive administered in a candy form selected from scents from the group which are added to the candy.
